(19) United States
(12) Patent Application Publication
(10) Pub. No.: US 2024/0329009 A1
(43) Pub. Date: Oct. 3, 2024

(54) Apparatus for pushing and pulling probe used for ultrasonic IRIS and eddy current testing

(71) Applicant: Guoxing Gu, Edmonton (CA)

(72) Inventor: Guoxing Gu, Edmonton (CA)

(21) Appl. No.: 18/600,501

(22) Filed: Mar. 8, 2024

Related U.S. Application Data
(60) Provisional application No. 63/455,329, filed on Mar. 29, 2023.

(51) Int. Cl. G01N 29/265; G01N 27/9013; G01N 33/2045

(52) U.S. Cl. CPC G01N 29/265; G01N 27/902; G01N 33/2045

(57) ABSTRACT

A handhold apparatus is provided for pushing in and pulling out an inspection probe to and from a tube of a heat exchanger for ultrasonic internal rotary inspection system (IRIS) and eddy current testing (ECT). The apparatus is light weight (less than 5 lb) and is wireless controlled for ease of use and portability. The probe can be moved forward, stop and backward via wireless control. There are both fine and coarse adjustment for the probe's moving speed as well as position limit setting and resetting as the probe's soft start point and soft end point for inspecting the first tube, subsequent inspections of other similar tubes in a bundle can be carried out quickly using the settings.

### FIELD OF THE INVENTION

The present invention relates to a probe pusher and puller or to a probe inserting and extracting apparatus, and more particularly, to a probe inserting and extracting apparatus for non destructive heat exchanger tubing inspections, such as ultrasonic internal rotary inspection system (IRIS) and eddy current testing (ECT) including near field array (NFA) inspection and remote field testing (RFT).

### BACKGROUND

A heat exchanger is a system used to transfer heat between a source and a working fluid. Heat exchangers are used in both cooling and heating processes. The fluids may be separated by a solid wall to prevent mixing or they may be in direct contact. They are widely used in space heating, refrigeration, air conditioning, power stations, chemical plants, petroleum refineries, petrochemical plants, natural gas processing, and sewage treatment. There are different types of heat exchangers, shell and tube heat exchanger is a common type. Heat transfer tubes are generally made of corrosion resistant metals, defects such as corrosion and erosion may occur on the inner and outer surfaces, they are evaluated for their integrity through non-destructive testing (NDT). Generally, IRIS, ECT, NFA and RFT are used for different application scenarios. A probe connected to a poly cable front end is inserted into a heat transfer tube, the probe is pushed in or pulled out manually or using a probot automatically. For IRIS operation, the probe is pushed in and pulled out slowly, as a general rule, at a speed of 1-2 inch/s while the data is acquired and recorded. The ultrasonic beam allows detection of metal loss from the inside and outside of the tube wall. While for eddy current testing, the probe is pushed in and pulled out at a wide range of speed depending on the method used.

Eddy current testing is one of many electromagnetic testing methods used in nondestructive testing making use of electromagnetic induction to detect and characterize surface and sub-surface flaws in conductive materials. Tubing inspection is generally limited to non-ferromagnetic tubing and is known as conventional eddy current testing (ECT). Conventional ECT is used for inspecting steam generator tubing in nuclear plants and heat exchangers tubing in power and petrochemical industries. To circumvent some of the shortcomings of conventional ECT, other eddy current testing techniques were developed with various successes, e.g. eddy current array (ECA), remote field testing (RFT), near field testing (NFT), remote-field array (RFA), near-field array (NFA), magnetic flux leakage (TFL), partial saturation eddy current (PSEC). There is a significant difference in speed of moving inspection probe for different eddy current methods, as a general rule, 4-20 inch/s for RFT with different excitation frequencies.

Brushless DC motors can produce stable torque from low speed to high speed. The maximum speed of brushless DC motors can reach several thousand to several tens of thousands of revolutions per minute (rpm). Hence, they are used for eddy current inspection systems. The higher the pushing or pulling probe speed the greater the motor torque required, the heavier the system to be. To cope with high speed operation requirement, heavy duty motors were used, which inevitably results in a heavy system that is inconvenient for an inspector to carry the system around working sites, especially when a working site is on a scaffold. Some of the portable, but heavy equipments are examples as below,
1. Probot: Drive 52 lb, take-up reel 14 lb, total 66 lb,
https://eddyfi.com/
2. High-Speed 3D Probe Pusher,
https://www.zetec.com/,
3. CoreStar TrackDrive-200 Probe Pusher: Main drive 46 lb, take-up drive 27 lb, OMNI-200 spool 6 lb, drive control pod 13 lb, total 92 lb,
https://www.corestar-corp.com/
4. USHER: Pusher drive unit 35.3 lb, take up reel drive unit 37.5 lb, total 72.8 lb,
https://www.inetec.hr/
5. Probe Push Puller HPP-2.V3: Probe drive mechanism 8.8 lb, SMC-2 motor control device 12.1 lb, total 20.9 lb,
https://www.eddymax.com/pushpull.htm/

U.S. Pat. No. 7,733, 084 B1 disclosed an eddy current acquisition system typically comprises an acquisition instrument, a probe cable slip ring, a probe, a probe adapter, a probe pusher, a take up reel, a probe pusher power supply, a probe pusher controller and in the case of inspection techniques requiring rotating probe technologies, a probe motor controller. The commercial product of this integrated system is similar to the High-Speed 3D Probe Pusher (https://www.zetec.com/). KR101476046B1 described a probe insertion and extraction apparatus for eddy current test. The probe insertion and extraction apparatus for eddy current test comprises: a cable reel for reeling in and storing a cable; a driving wheel which extracts the cable from the cable reel; a driving motor which generates torque for driving the cable reel and the driving wheel. CN107677725A also described an eddy current probe single shaft pushing away and pulling out device.

All of above mentioned products and disclosures are for eddy current testing and they can also be used for ultrasonic IRIS, however, they are heavy and not optimized for IRIS. In real practices, inspectors would like to push and pull probes manually without using the heavy equipments. Manual push and pull can move probe for 1-2 feet at a time and can not maintain a constant probe moving speed. It is highly desirable to develop a handhold probe pusher and puller with light weight, preferably less than 5 lbs.

A high torque is normally required at the start of a motion and significant lower torque is required thereafter for continuous motion. Stepper motors can provide a relatively large torque, especially at low speeds. For the same motor frame size and weight, stepper motors can produce five times of torque compared with brushless DC motors at a low speed. Stepper motors are the best choice for generating torque required for a device with very light weight, preferably less than 5 lb.

In order to reduce the weight, in present invention, a light weight stepper gearmotor is used for moving the probe precisely at a desired speed, the whole apparatus is primarily made of aluminum and plastics forming a very compact design and light weight in less than 5 lb for ease of use and portability. The light weight stepper gearmotor can provide high torque at a low speed for IRIS testing and reduced torque at high speed still good for eddy current inspections.

Another object of the present invention is to minimize or even eliminate flow back water in IRIS operation, avoiding back splashing water from wetting operators.

### SUMMARY OF THE INVENTION

A handhold apparatus is provided for pushing in and pulling out an inspection probe to and from a tube of a heat exchanger for ultrasonic internal rotary inspection system (IRIS) and eddy current testing (ECT) operations. The inspection probe having a sensor at its front end is connected to a data acquisition system via a long poly cable, so the apparatus is actually for pushing in and pulling out the poly cable to and from a tube of a heat exchanger, as a result, inspection probe and inspection cable are interchangeably used or even simplified as probe or cable in the specification. The apparatus is wireless controlled for moving the inspection probe forward, stop and backward. There are both fine and coarse adjustment for the probe's moving speed as well as position limit setting and resetting as a probe's soft start point and soft end point for inspecting the first tube, subsequent inspections of other similar tubes in a bundle can be carried out quickly using the settings. The apparatus comprises a main structure, a top pulley assembly, an electronic control system, a supporting tube assembly, a timing belt, a timing belt tension adjustment mechanism, a hollow plastic handle and a water gun. The main structure comprises a square tube, a gearmotor, a driver timing pulley, a driven timing bearing pulley, one or two supporting timing bearing pulleys, a tube adapter, a front plate and a rear plate. A timing bearing pulley is a pulley with teeth evenly allocated on the circumferential surface and a bearing in the center. A bearing pulley is a pulley with only a bearing in the center. The top pulley assembly having three or four bearing pulleys arranged in series, which can be adjusted to move close and away from the main structure for tightening or loosening the grip of the inspection cable, which has a probe at the front end. The electronic control system comprises a motor driver integrated circuit (IC), a wireless motor controller, a motor speed adjustment dial, a power entry jack for connecting a DC power supply, a momentary trigger switch and a LED indicator, a separated wireless remote key pad with three or four keys and a control program. The motor driver IC and the wireless motor controller can be either enclosed in an electronic control box that is attached to one side of the square tube of the main structure or directly enclosed in the square tube or the hollow plastic handle to make the apparatus highly compact and light weight. The timing belt tension adjustment mechanism can be a separated “C” clamp with a bearing pulley or a slot on the main structure. The “C” clamp is inserted upward into a slot at the bottom of the main structure for tightening the timing belt. The supporting tube assembly is also attached to the main structure comprising multiple tubes and 90 degree clamps for three dimensional adjustments required for positioning the apparatus. The supporting tube assembly can be replaced by either a uniport or a triport as well.

### BRIEF DESCRIPTION OF THE DRAWINGS

FIG. 1 is a photo of the apparatus for pushing and pulling the inspection cable with three pairs of pulleys for grip of the cable.

FIG. 2 is a photo of top view of the apparatus for pushing and pulling the inspection cable with three pairs of pulleys for grip of the cable.

FIG. 3 is a schematic illustration of a timing belt tension adjustment mechanism comprising a “C” clamp and a bearing pulley.

FIG. 4 is a view of its control panel including a motor speed adjustment dial, a power jack, a momentary trigger switch and a LED indicator as well as a separated wireless remote key pad with 3 keys or 4 keys.

FIG. 5 is an example of digital inputs for settings and operation using a remote key pad with 3 keys in various combinations for the control program.

FIG. 6 is a schematic illustration of a variation of the apparatus with 4 pairs of pulleys for grip of the inspection cable.

FIG. 7 is a schematic illustration of a variation of the apparatus without the side attached electronic control box.

FIG. 8 is a schematic illustration of one side of the main structure showing the holes and slots for installations of the driver timing pulley, the driven timing bearing pulley, the two supporting timing bearing pulleys and the timing belt tension adjustment bearing pulley.

FIG. 9 is a schematic illustration of the front plate and the rear plate attached to the main structure.

FIG. 10 is a schematic illustration of the water gun for an ultrasonic internal rotary inspection system.

FIG. 11 is a schematic illustration of a long insertion tube and a short insertion tube adapter.

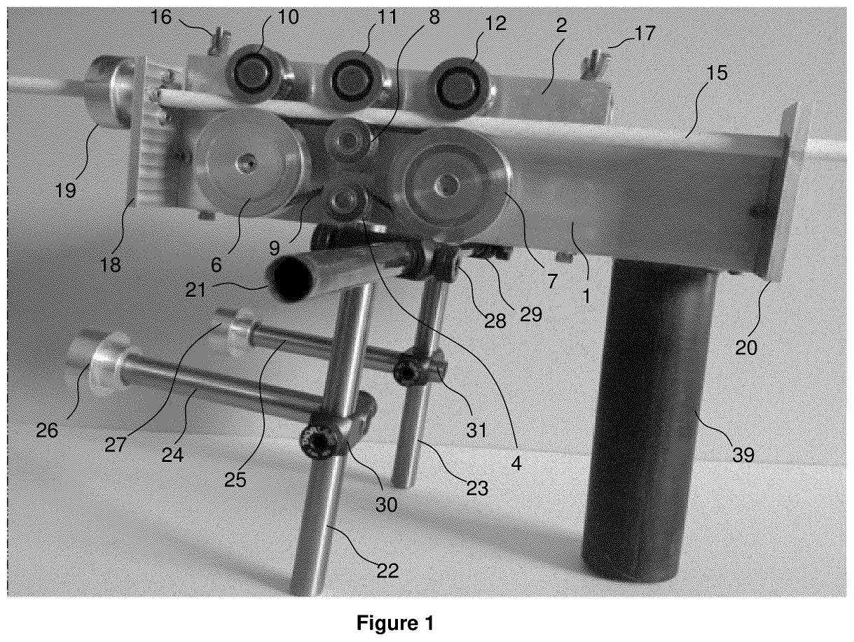

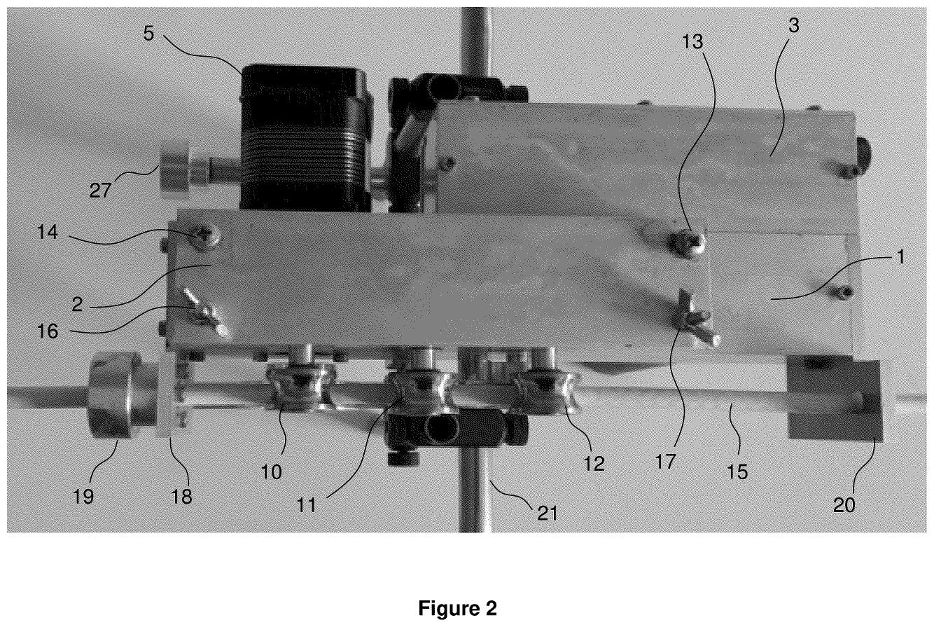

The present invention is described below with reference to FIGS. 1 and 2, which are two photos of the apparatus for pushing and pulling inspection cable with three pairs of pulleys for grip of the inspection cable, comprising a main structure, a top pulley assembly (2), an electronic control system, a timing belt (9) and a timing belt tension adjustment mechanism with a bearing pulley (4), a supporting tube assembly and a hollow plastic handle (39). The main structure comprise an square tube (1), a motor (5), a driver timing pulley (6), a driven timing bearing pulley (7), and a supporting bearing pulley (8). The motor is either a stepper gearmotor or a brushless DC gearmotor, preferably a stepper gearmotor having a gear ratio of 1-15:1. The driver timing pulley (6) is firmly attached to the motor (5), providing torque to move all other pulleys (4, 6, 7 and 8) that are arranged on the same side of the square tube (1). The top pulley assembly (2) having three grooved bearing pulleys (10, 11 and 12), two sliding guiders (13 and 14) and two adjustment screws with butterfly nuts (16 and 17). The three grooved bearing pulleys are arranged on the same side in series, the sliding guiders and the adjustment screws are perpendicular to the shafts of the pulleys. The whole pulley assembly (2) can be adjusted to move close to and away from the main structure for tightening or loosening the grip of the inspection cable (15) via adjustments of the butterfly nuts (16 and 17). The main structure also comprises a front plate (18) with an attached tube adapter (19) and a rear plate (20) for keeping the inspection cable (15) in the center of the upper and the lower pulley pairs. The supporting tube assembly is attached to the main structure for three dimensional adjustments so that the apparatus can be positioned firmly for tube inspection; it comprises a horizontal tube (21) attached to the very bottom of the main structure, two vertical tubes (22 and 23) and another two horizontal tubes (24 and 25) with tube adapters (26 and 27) assembled using 90 degree clamps (28, 29, 30 and 31) such that they can be adjusted in three dimensions flexibly. Each of the tube adapters is for connecting two tubes with different diameters. The tube adapter (19) can also be called reducer, e.g. 1.0 inch to 0.5 inch, 1.0″ to 1.50″, etc. Different tube reducers can be used for inspecting tubes with different sizes.

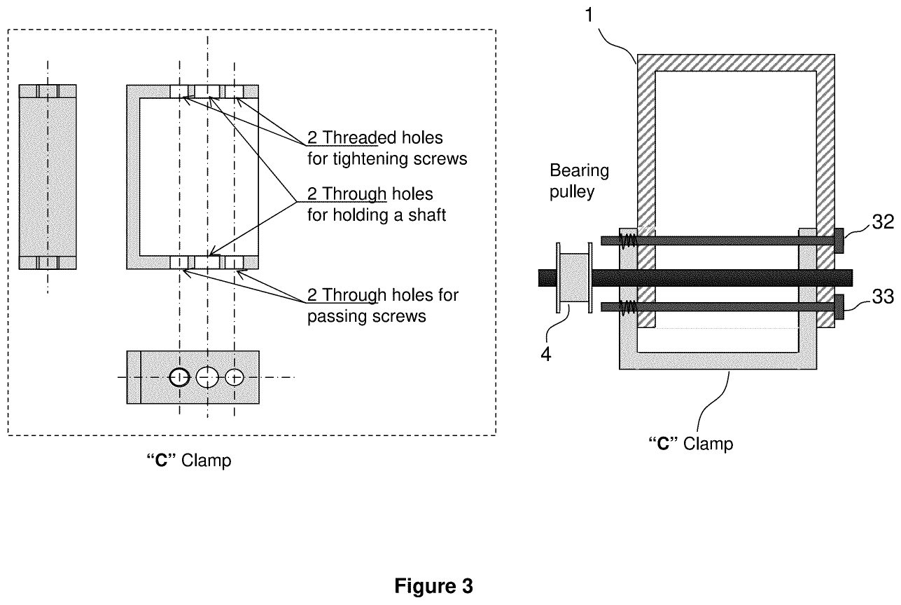

FIG. 3 is a schematic illustration of a timing belt tension adjustment mechanism, comprising a “C” clamp and a bearing pulley (4). The “C” clamp comprises a short channel, one shaft and two adjustable screws (32 and 33). The bearing pulley (4) is secured on the shaft. The “C” clamp with the bearing pulley (4) can slide upward into a slot (not shown) at the bottom of the square tube (1) and secured at a position via adjusting the two crews (32 and 33) so that the timing belt (9) has a proper belt tension.

The electronic control system comprises a motor driver IC, a wireless motor controller wired to the motor driver IC, a motor speed adjustment dial (34), a power entry jack (35), a momentary trigger switch (36) and a LED indicator (37), a separated wireless remote key pad with either three keys (38a) or four keys (38b) and a control program. The motor driver IC and the wireless motor controller can be either enclosed in an electronic control box (3) that is attached to one side of the square tube (1) of the main structure or directly enclosed in the square tube (1) or the hollow plastic handle (39) to make the apparatus highly compact and light weight.

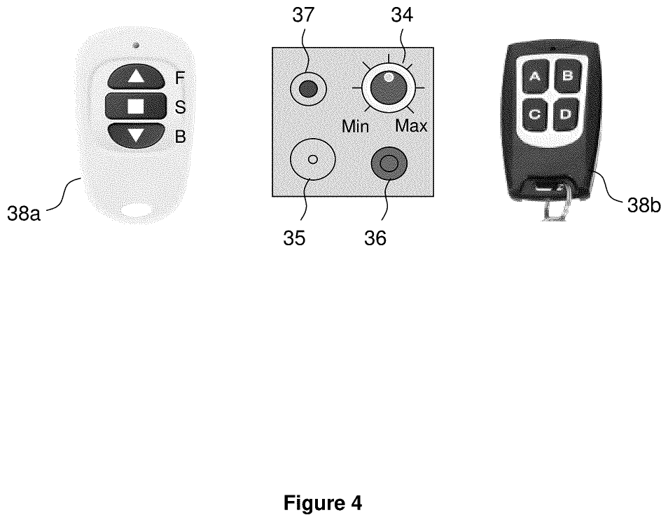

FIG. 4 is a view of its electronic control panel of the electronic control system, including a motor speed adjustment dial (34), a power entry jack (35) for connecting a DC power supply (9-28 V), a momentary trigger switch (36) and a LED indicator (37) as well as a separated wireless remote key pad with three keys (38a): Forward (F), Stop(S) and Backward (B), or four keys (38b, A, B, C and D). The electronic control panel is on the electronic control box (3) when the motor driver IC and the wireless motor controller are enclosed in the electronic control box. The electronic control panel is on the rear plate (20) of the main structure if the motor driver IC and the wireless motor controller are directly enclosed in the square tube (1) or the hollow plastic handle (39). The wireless remote key pad gives instructions (F, S and B for the tree key pad (38a); A, B, C and D for the four key pad (38b) to turn the motor clockwise or counter-clockwise (move the inspection cable forward or backward), the momentary trigger switch (36) for functional change, the motor speed adjustment dial (34) for motor speed control and a LED indicator (37) for functional status indication.

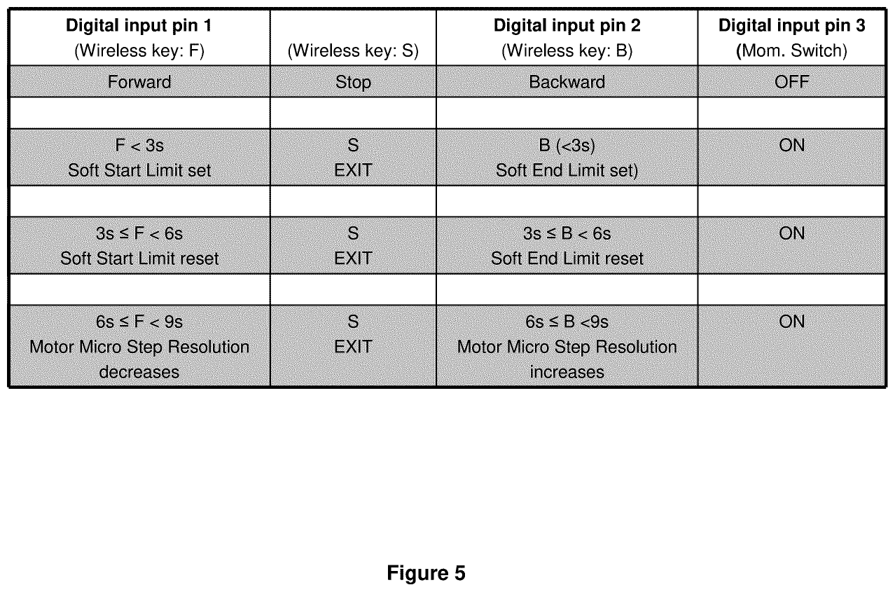

FIG. 5 is an example of digital inputs for settings and operation using a remote key pad with 3 keys in various combinations for the control program. Referring to FIGS. 4 and 5, as an example of the three keys (38a) on the wireless keypad, it can be used as multifunctional keys in conjunction with the momentary trigger switch (36) for motor directional control, soft limit setting and resetting as well as motor micro step control to shift the motor speed from one range to another range. The motor speed adjustment dial (34) is connected to an analog input pin 0 (IN_0), the LED indicator (37) to a digital output pin 0 (OUT_0), and the momentary trigger switch (36) to the digital input pin 3 (IN_3) of the motor driver IC respectively. The Forward (F) key and Backward (B) key are wirelessly connected to digital input pin 1 (IN_1) and digital input pin 2 (IN_2) of the motor driver IC respectively via the wireless motor controller. The three keys on the wireless keypad are for the motor directional toggle control, forward, stop and backward, when the momentary switch is not triggered. The three wireless keys are for the motor soft start and stop limit settings when the momentary switch is triggered and the key is pressed for less than 3 seconds, where F key is for entering the motor soft start limit setting, B key is for entering the motor soft end limit setting, S key is for exiting a setting. The three wireless keys are for the motor soft limit resetting when the momentary switch is triggered and the key is pressed for equal or greater than 3 seconds and less than 6 seconds, where F key is for entering soft start limit resetting, B key is for entering soft end limit resetting, S key is for exiting a resetting. The three wireless keys are for the motor micro step resolution control to shift the motor speed from one range to another range when the momentary switch is triggered and the key is pressed for equal or greater than 6 seconds and less than 9 seconds, where F key is for entering the micro step resolution decrease setting, B key is for entering micro step resolution increase setting, S key is for exiting a setting.

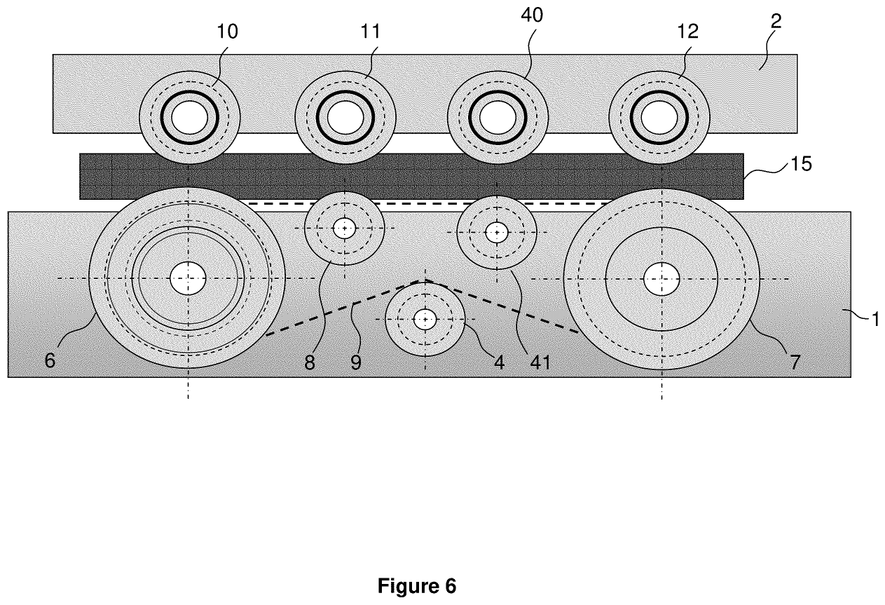

FIG. 6 is a schematic illustration of a variation of the apparatus with 4 pairs of pulleys for grip of the inspection cable, one additional pair of pulleys is a grooved bearing pulley (40) mounted on the top pulley assembly (2) and a timing bearing pulley (41) mounted on the square tube (1) of the main structure.

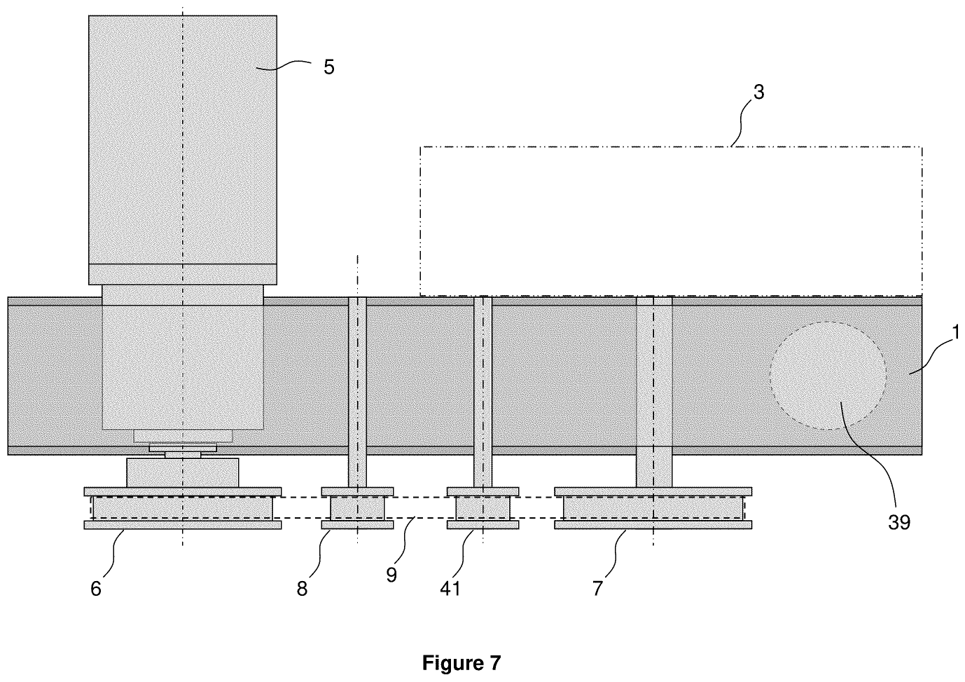

FIG. 7 is a schematic illustration of a variation of the apparatus without the side attached electronic control box (3), where all electronic components (not shown in FIG. 7) are installed inside of the square tube (1) of the main structure or inside of the hollow plastic handle (39) forming a highly compact apparatus.

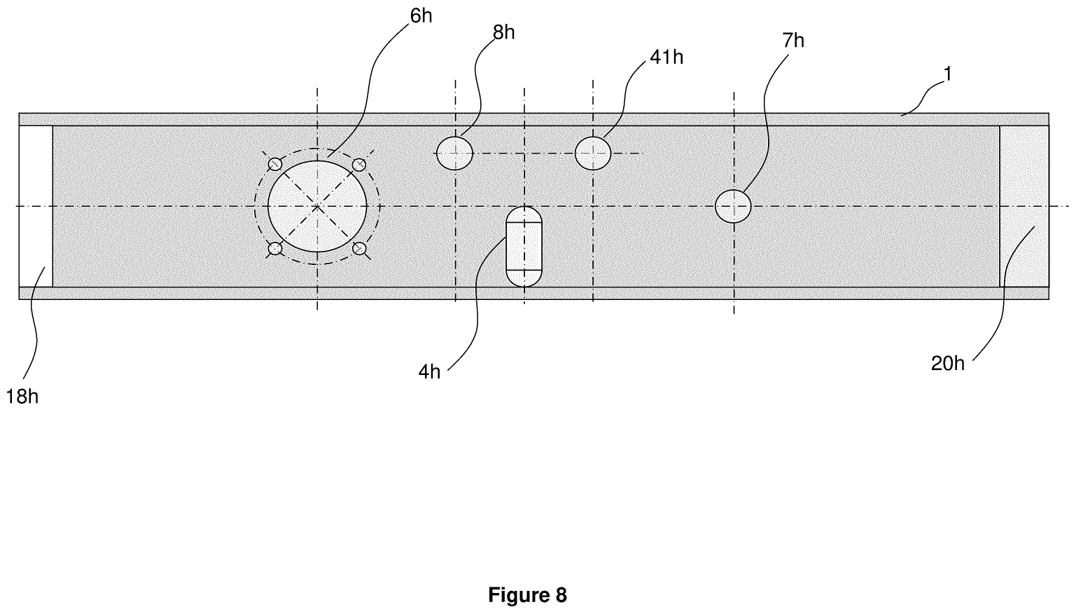

FIG. 8 is a schematic illustration of one side of the main structure showing the holes and slots for installations of the driver timing pulley (6h), the driven timing bearing pulley (7h), the two supporting timing bearing pulleys (8h and 41h) and the timing belt tension adjustment bearing pulley (4h) as well as the front plate (18h) and the rear plate (20h).

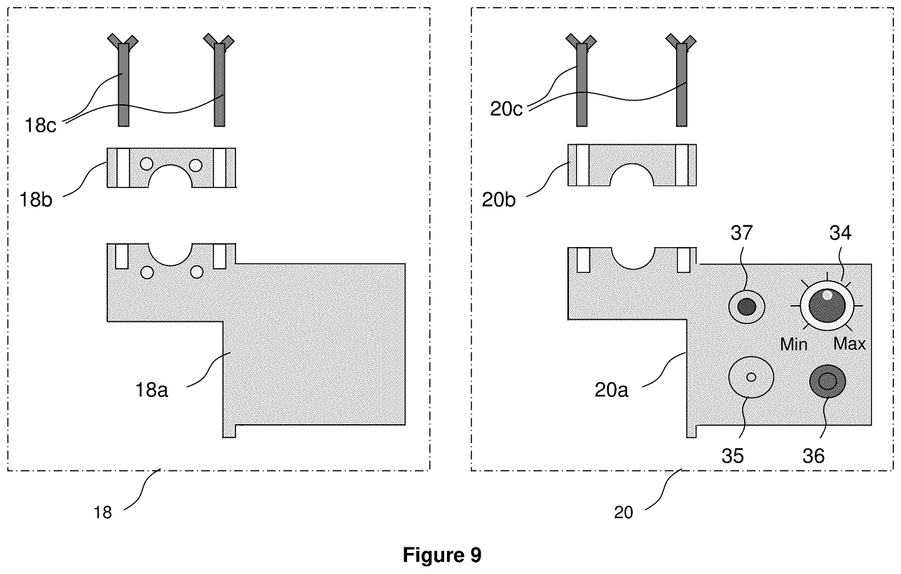

FIG. 9 is a schematic illustration of the front plate (18) and the rear plate (20) attached to the main structure at the slots (18h) and (20h). The front plate (18) comprises a lower part (18a) with a half circular slot, 2 threaded vertical holes and 2 facing through holes on the left side and bare plate on the right side, and the upper part (18b) with a half circular slot, 2 vertical through holes and 2 facing through holes, as well as two butterfly screws (18c) that can screw-tighten the lower part and the top part into an integrated front plate (18). The rear plate (20) comprises a lower part (20a) with a half circular slot and 2 threaded vertical holes on the left side and electrical panel on the right side, and the upper part (20b) with a half circular slot and 2 vertical through holes, as well as 2 butterfly screws (20c) that can screw-tighten the lower part (20a) and the top part (20b) into an integrated rear plate (20). The two half circular slots on the front plate form a half-circular-slot-forming-hole for holding and guiding the inspection cable (15) when the two butterfly screws (18c) are tightened up, and so do the two half circular slots on the rear plate, whereby the inspection cable (15) can be flexibly inserted or removed from the holes. The other four facing through holes on the front plate (18) can be used to attach the tube adapter (19).

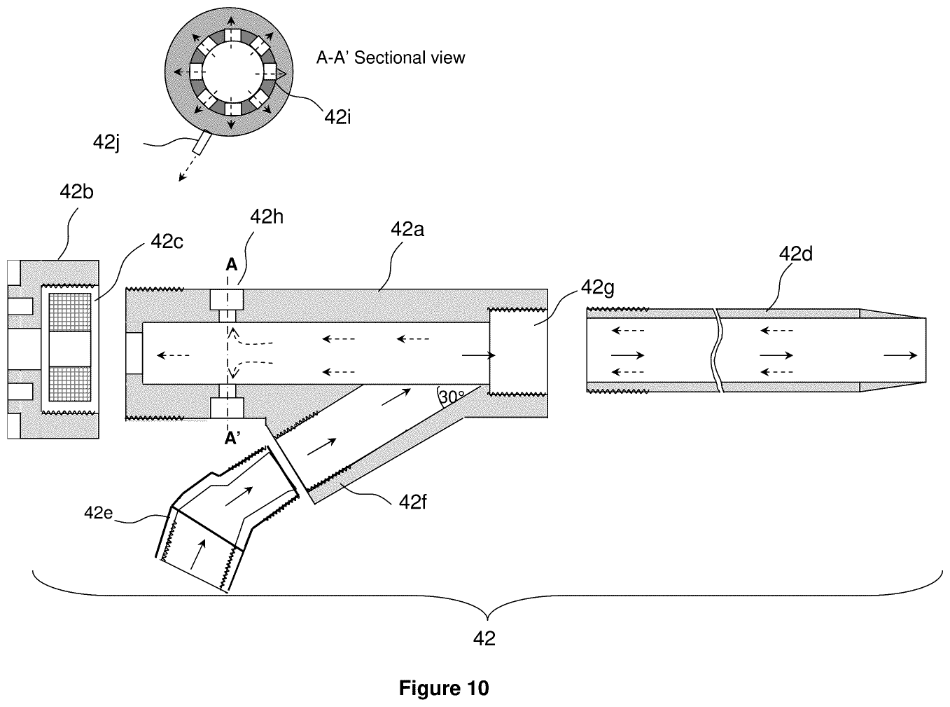

FIG. 10 is a schematic illustration of the water gun (42) for an ultrasonic internal rotary inspection system, where the water gun (42) comprises a “Y” shape three-way fitting (42a), a back adapter (42b), a sponge ring (42c), a long insertion tube (42d) with a male thread at the back end and a cone shape at the front end, and a 45° male-to-female fitting (42e). The “Y” shape fitting's side branch inlet (42f) is approximately 30° inclined to the straight body, which has male-threaded left side for connecting the back adapter (42b) and female-threaded right side (42g) for connecting the long insertion tube (42c), and a water trap-and-drain mechanism (42h) as shown in the A-A sectional view in details. The water trap-and-drain mechanism comprises 8 through holes evenly allocated in a circumferential groove, which is circumferentially covered using a circular strip, and a water drain outlet on the circular strip. It allows the flow back water (dashed arrows) to enter the circumferential groove via the eight holes (42i), mix in the groove and then drain out at the outlet (42j). The back adapter can be screwed on to the left side of the “Y” fitting, compressing the sponge (42e) to form a seal. The water gun can then be attached to the front plate (18) using 4 facing through screws, replacing the tube adapter (19). When all parts are connected to form the water gun, the flooding water stream enters the water gun at the inlet of the 45° male-to-female fitting (42e) and flows into the long insertion tube (42d) as indicated by the solid arrows, and flows back and enters the trap-and-drain mechanism (42h) and finally drain out at the outlet (42j) as indicated by the dashed arrows.

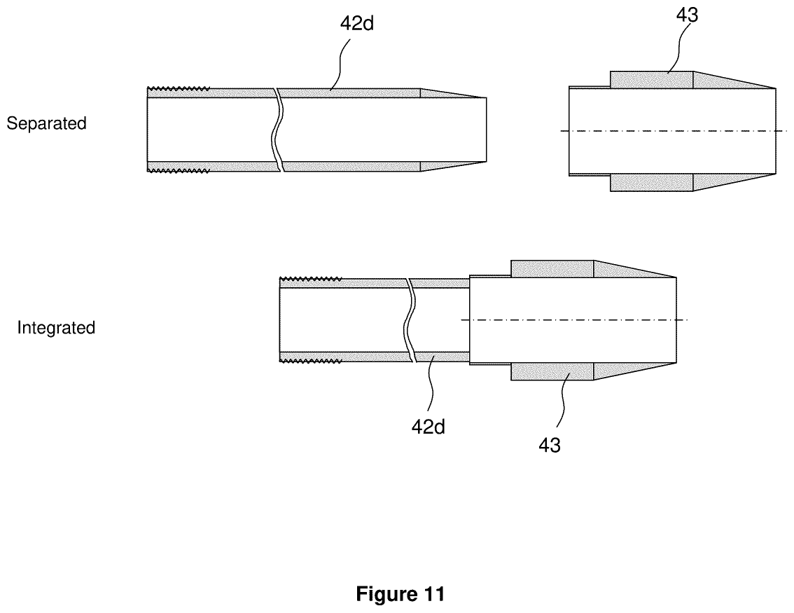

FIG. 11 is a schematic illustration of the long insertion tube (42d) with a short insertion tube adapter (43) with a grooved back end and a larger cone shapes front end. The long insertion tube (42d) is for 1.0-1.25″ tube inspection, and can be inserted into the short insertion tube adapter (43) to form an integrated insertion tube as shown at the bottom part of the drawing for 1.5-2.0″ tube inspection, whereby eliminating the use of a larger size insertion tube and reducing the overall equipment weight. The deeply grooved back end of the short insertion tube adapter is for application of a tape to secure it onto the long insertion tube.

It is to be understood by those skilled in the art that the present invention may be embodied in many other forms without departing from the spirit and scope of the invention, Accordingly, the above-described embodiments are to be considered illustrative and not restrictive, and all embodiments falling within the scope of the appended claims and their equivalents are intended to be included within the scope of the present invention.

The invention claimed is:

1. The apparatus comprises a main structure, a top pulley assembly, an electronic control system, a timing belt, a timing belt tension adjustment mechanism, a hollow plastic handle, a supporting tube assembly and a water gun.

2. The apparatus of claim 1, wherein the main structure comprises
a square tube,
a motor being either a stepper gearmotor or a brushless DC gearmotor, preferably a stepper gearmotor having a gear ratio of 1-15:1,
a driver timing pulley firmly attached to the motor that is secured on the square tube,
a driven timing bearing pulley and one or two supporting timing bearing pulleys all firmly attached to the square tube,
a tube adapter,
a front plate attached to the main structure at the front of the square tube comprises
a lower part with a half circular slot, 2 threaded vertical holes and 2 facing through holes on the left side and bare plate on the right side,
an upper part with a half circular slot, 2 vertical through holes and 2 facing through holes, and
two butterfly screws that can screw-tighten the lower part and the top part into an integrated front plate;
wherein the other four facing through holes on the front plate can be used for attaching the tube adapter, and
a rear plate attached to the main structure at the end of the square tube comprising
a lower part with a half circular slot and 2 threaded holes on the left side and electrical panel on the right side,
an upper part with a half circular slot and 2 through holes, and
two butterfly screws that can screw-tighten the lower part and the top part into an integrated rear plate;
wherein the two half circular slots on the front plate form a half-circular-slot-forming-hole for holding and guiding the inspection cable when the two butterfly screws are tightened up, and so do the two half circular slots on the rear plate, whereby the inspection cable can be flexibly inserted or removed from the holes.

3. The apparatus of claim 1, wherein the top pulley assembly comprises
three or four bearing pulleys arranged on the same side in series,
two sliding guiders, and
two adjustment screws with butterfly nuts;
wherein both the sliding guiders and the adjustment screws are perpendicular to the shafts of the bearing pulleys; and
wherein the top pulley assembly can be adjusted to move close to and away from the main structure for tightening or loosening the grip of an inspection cable via adjustments of the butterfly nuts.

4. The apparatus of claim 1, wherein the electronic control system comprises
a motor driver integrated circuit (IC),
a wireless motor controller wired to the motor driver IC;
wherein the motor driver and the wireless motor controller can be either enclosed in an electronic control box that is attached to one side of the square tube of the main structure or directly enclosed in the square tube or the hollow plastic handle to make the apparatus highly compact and light weight,
an electronic control panel comprising
a motor speed adjustment dial,
a power jack for connecting a DC power supply (9-28 V),
a momentary trigger switch as a multifunctional key, and
a LED indicator;
wherein the electronic control panel is on the electronic control box when the motor driver and the wireless motor controller are enclosed in the electronic control box; and
wherein the electronic control panel is on the rear plate of the main structure if the motor driver and the wireless motor controller are directly enclosed in the square tube or in the hollow plastic handle,
a separated wireless remote key pad with three keys: Forward (F), Stop(S) and Backward (B), or a separated wireless remote key pad with four keys: A, B, C and D, and
a control program.

5. The apparatus of claim 4, as an example of using a wireless remote key pad with 3 keys: Forward (F), Stop(S) and Backward (B),
wherein the motor speed adjustment dial is connected to an analog input pin 0 (IN_0), the LED indicator to a digital output pin 0 (OUT_0), and the momentary trigger switch to the digital input pin 3 (IN_3) of the motor driver IC respectively; and
wherein the Forward (F) key and Backward (B) key are wirelessly connected to digital input pin 1 (IN_1) and digital input pin 2 (IN_2) of the motor driver IC via the wireless motor controller; and
wherein the control program uses the three keys (F, S and B) on the wireless remote key pad in conjunction with the momentary trigger switch for motor directional control, motor soft limit setting and resetting as well as motor micro step resolution control to shift the motor speed from one range to another range; and
wherein the three keys (F, S and B) are programmed for
a. motor directional toggle control, forward, stop and backward, when the momentary switch is not triggered,
b. motor soft limit settings when the momentary switch is triggered and the key is pressed for less than 3 seconds,
c. motor soft limit resettings when the momentary switch is triggered and the key is pressed for equal or greater than 3 seconds and less than 6 seconds, and
d. motor micro step resolution control to shift the motor speed from one range to another range when the momentary switch is triggered and the key is pressed for equal or greater than 6 seconds and less than 9 seconds.

6. The apparatus of claim 1, wherein the timing belt tension adjustment mechanism comprising
a “C” clamp comprises a short channel, one shaft, and two adjustable screws, and
a bearing pulley secured on the shaft;
wherein the bearing pulley can slide upward into a slot at the bottom of the square tube and secured at a position via adjusting the two crews so that the timing belt has a proper belt tension.

7. The apparatus of claim 1, wherein the supporting tube assembly comprises
a horizontal tube attached to the very bottom of the square tube,
two vertical tubes, and
another two horizontal tubes with tube adapters;
wherein all the tubes are assembled using 90 degree clamps such that they can be adjusted in three dimensions flexibly so that the apparatus can be positioned firmly for tube inspection.

8. The apparatus of claim 2, wherein the tube adapter can be a tube reducer, e.g. 1.0″ to 0.5″, 1.0″ to 1.5″ etc., and different tube reducers can be used for inspection of tubes with different sizes.

9. The apparatus of claim 1, wherein the water gun used for an ultrasonic internal rotary inspection system (IRIS) comprises
a “Y” shape three-way fitting,
a back adapter working as the tube adapter,
a sponge ring sitting in the back adapter,
a 45° male-to-female fitting,
a long insertion tube with a male thread at the back end and a cone shape at the front end,
a short insertion tube adapter with a grooved back end and a larger cone shapes front end;
wherein the long insertion tube's front end can be inserted into the short insertion tube adapter, forming an integrated insertion tube for larger size tube inspection; and
wherein the deeply grooved back end of the short insertion tube adapter is for application of a tape to secure it onto the long insertion tube.

10. The apparatus of claim 9, wherein the “Y” shape fitting comprises
a side branch inlet being approximately 30° inclined to the straight body for connecting the 45° male-to-female fitting,
a male-threaded left side for connecting the back adapter,
a female-threaded right side for connecting the long insertion tube, and
a water trap-and-drain mechanism comprising
8 through holes evenly allocated in a circumferential groove, which is circumferentially covered using a circular strip, and
a water drain outlet on the circular strip.

11. The apparatus of claim 9, wherein the back adapter is screwed into the left side of the “Y” fitting, compressing the sponge in the back adapter to form a seal, and the long insertion tube is screwed into the right side of the “Y” fitting, the water gun can then be attached to the front plate at the four facing through holes using 4 screws, replacing the tube adapter.
